(19) United States
(12) Patent Application Publication
(10) Pub. No.: US 2009/0088460 A1
(43) Pub. Date: Apr. 2, 2009

(54) Administration of E-3[3-[N-(4-methoxybenzenesulfonyl)-N-isopropylamino]phenyl]-3-(3-pyridyl)propenohydroxamic acid for preventing and/or treating inflammatory cutaneous pathologies/afflictions

(75) Inventor: Thibaud Biadatti, Opio (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH &DEVELOPMENT, BIOT (FR)

(21) Appl. No.: 12/232,457

(22) Filed: Sep. 17, 2008

(30) Foreign Application Priority Data
Mar. 16, 2007 (FR) PCT/FR2007/050940

(51) Int. Cl. A61K 31/4406

(52) U.S. Cl. 514/357

(57) ABSTRACT

Inflammatory cutaneous pathologies/afflictions, e.g., eczema or psoriasis, are prevented and/or treated by administering to a subject in need of such treatment, a thus effective amount of E-3-[3-[N-(4-methoxybenzenesulfonyl)-N-isopropylamino]phenyl]-3-(3-pyridyl)propenohydroxamic acid of formula (I):

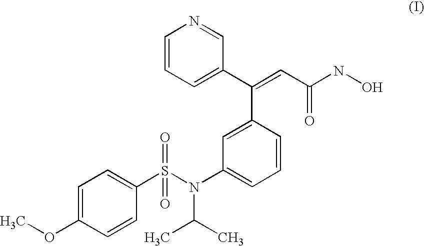

or of a pharmaceutically acceptable salt, solvate or hydrate thereof.

### CROSS-REFERENCE TO COMPANION APPLICATION

Co-pending U.S. patent application Ser. No. ______ [Attorney Docket No. 1034227-000940], filed concurrently herewith, hereby expressly incorporated by reference and assigned to the assignee hereof.

### CROSS-REFERENCE TO PRIORITY/PCT APPLICATIONS

This application claims priority under 35 U.S.C. § 119 of FR 06/02428, filed Mar. 20, 2006, and is a continuation/national phase of PCT/FR 2007/050940, filed Mar. 16, 2007 and designating the United States (published in the French language on Sep. 27, 2007 as WO 2007/107664 A2; the title and abstract were also published in English), each hereby expressly incorporated by reference in its entirety and each assigned to the assignee hereof.

### BACKGROUND OF THE INVENTION

1. Technical Field of the Invention

The present invention relates to the administration of E-3-[3-[N-(4-methoxybenzenesulfonyl)-N-isopropylamino]-phenyl]-3-(3-pyridyl)propenohydroxamic acid, or of a pharmaceutically acceptable salt, pharmaceutically acceptable solvate or hydrate thereof, in a regime or regimen for preventing and/or treating inflammatory cutaneous pathologies/afflictions, and particularly eczema or psoriasis.

2. Description of Background and/or Related and/or Prior Art

EP 1314721 describes a family of propenohydroxamic acid derivatives, which are TNF-alpha-converting enzyme inhibitors, and which can be used in the treatment or prevention of septicaemia, rheumatoid arthritis, osteoarthritis, infectious diseases, autoimmune diseases, malignant tumors, collagen diseases, chronic ulcerative colitis and insulin-independent diabetes.

### SUMMARY OF THE INVENTION

It has now unexpectedly been found that one of these compounds, namely, E-3-[3-[N-(4-methoxybenzene-sulfonyl)-N-isopropylamino]phenyl]-3-(3-pyridyl)propenohydroxamic acid, is active for preventing and/or treating inflammatory cutaneous pathologies/afflictions, and particularly eczema or psoriasis.

### BRIEF DESCRIPTION OF THE DRAWINGS

FIG. 1 is a graph showing the modulation of AA-induced mouse ear edema by indomethacin and by the compound (I) according to the invention, and

FIG. 2 is a graph showing the modulation of TPA-induced mouse ear edema by VdB and by the compound (I) according to the invention.

### DETAILED DESCRIPTION OF BEST MODE AND SPECIFIC/PREFERRED EMBODIMENTS OF THE INVENTION

The present invention thus features administration of at least one compound selected from the compound of formula (I) below:

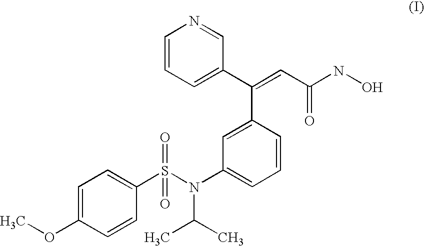

corresponding to Example 17(1) (compound 88) of EP 1314721, namely, E-3-[3-[N-(4-methoxybenzene-sulfonyl)-N-isopropylamino]phenyl]-3-(3-pyridyl)-propenohydroxamic acid, and subsequently referred to as compound (I), pharmaceutically acceptable salts thereof, pharmaceutically acceptable solvates thereof and hydrates thereof, in a regime or regimen for preventing and/or treating inflammatory cutaneous pathologies/afflictions. These pathologies are in particular all forms of psoriasis, whether cutaneous, mucosal or ungual, psoriatic arthritis, or else cutaneous atopy, such as eczema, or respiratory atopy or else gingival hypertrophy.

The pharmaceutically acceptable salts of the compound of formula (I) comprise those with mineral or organic acids such as hydrochloric acid, hydrobromic acid, sulfuric acid, phosphoric acid, nitric acid, picric acid, oxalic acid, sulfonic acids such as methanesulfonic acid, benzenesulfonic acid, p-toluenesulfonic acid, mesitylenesulfonic acid or naphthalenesulfonic acid, tartaric acid, dibenzoyl-tartaric acid, mandelic acid, acetic acid, propionic acid, lactic acid, citric acid, succinic acid, fumaric acid, maleic acid, malonic acid, phthalic acid or camphosulfonic acid, and preferably acetic acid, maleic acid, citric acid, tartaric acid, methane-sulfonic acid, phosphoric acid, nitric acid, sulfuric acid, hydrobromic acid or hydrochloric acid.

The salts of the compounds of formula (I) also comprise salts with organic or mineral bases, for example the alkali metal salts, such as the lithium, sodium or potassium salts.

The expression “hydrate of a compound of formula (I)” means the combination of this compound with one or more molecules of water.

The expression “solvate of a compound of formula (I)” means the association resulting from the binding of a solvent to the crystals of compound of formula (I) that are formed in the presence of this solvent.

For use as a medicament, compound (I), or a pharmaceutically acceptable salt thereof or a pharmaceutically acceptable solvate or hydrate thereof, should be formulated in a pharmaceutical or dermatological composition.

The present invention therefore also features pharmaceutical, preferably dermatological, compositions comprising compound (I), or a pharmaceutically acceptable salt thereof, a pharmaceutically acceptable solvate thereof or a hydrate thereof, for the treatment and/or prevention of inflammatory cutaneous pathologies, and in particular all forms of psoriasis, whether cutaneous, mucosal or ungual, and even psoriatic arthritis, or else cutaneous atopy, such as eczema, or respiratory atopy or else gingival hypertrophy.

This invention also features pharmaceutical, preferably dermatological, compositions comprising compound (I), or a pharmaceutically acceptable salt thereof, a pharmaceutically acceptable solvate thereof or a hydrate thereof, for the treatment and/or prevention of eczema or psoriasis.

Such compositions are useful, and therefore suitable, for oral, topical, enteral, parenteral, ocular, sublingual, inhaled, subcutaneous, intramuscular, intravenous, transdermal, local or rectal administration. Compound (I), optionally in the form of a pharmaceutically acceptable salt, solvate and/or hydrate, alone or in combination with another active ingredient, can be administered in a unit administration form, or as a mixture with conventional pharmaceutical carriers or excipients, to animals and to humans. Preferably, the pharmaceutical composition is packaged in a form suitable for oral or topical administration.

The compositions according to the invention contain compound (I), or a pharmaceutically acceptable salt, pharmaceutically acceptable solvate or hydrate thereof, in a sufficient amount to obtain the desired prophylactic or therapeutic effect. The posology that can be used varies according to the age, sex and weight of the patient. Compound (I), or a salt, solvate or hydrate thereof, will preferably be administered at a rate of from 0.01 to 100 mg/kg per day, advantageously from 0.01 to 50 mg/kg per day. It is also possible to administer such doses in 2 to 4 daily administrations. Although these dosages are examples of average situations, there may be specific cases where higher or lower dosages are appropriate; such dosages are also according to the invention.

The compositions according to the invention comprise a physiologically acceptable carrier or at least one pharmaceutically acceptable excipient, selected according to the desired pharmaceutical, preferably dermatological, form and the selected method of administration.

The expression “physiologically acceptable carrier and pharmaceutically acceptable excipient” means, respectively, a carrier and an excipient that are compatible with the skin, the mucous membranes and the appendages.

For oral administration, the pharmaceutical or dermatological composition may be in the form of tablets, gel capsules, dragees, pills, syrups, suspensions, solutions, powders, granules, emulsions, capsules, or lipid or polymeric microspheres, nanospheres or vesicles for controlled release. For parenteral administration, the composition may be in the form of solutions or suspensions for infusion or for injection. To obtain a solid composition for oral administration, the active ingredient may be mixed with at least one inert diluent, such as sucrose, lactose or starch. In general, other additives, such as a lubricant, for instance magnesium stearate, may be added. In the case of capsules, tablets or pills in particular, a buffer may be added. In the case of liquid oral compositions, an inert diluent such as water may be used.

Administered topically, the pharmaceutical composition according to the invention is more particularly useful for treating the skin and the mucous membranes and may be in the form of salves, creams, milks, ointments, powders, impregnated pads, syndets, solutions, gels, sprays, foams, suspensions, lotions, sticks, shampoos or washing bases. It may also be in the form of suspensions of lipid or polymeric microspheres, nanospheres or vesicles or polymeric patches and hydrogels for controlled release. This composition for oral administration may be in anhydrous form, in aqueous form or in the form of an emulsion.

When it is administered orally, compound (I), or a salt, solvate or hydrate thereof, is administered at a rate of from 0.01 to 100 mg/kg per day, advantageously from 0.01 to 50 mg/kg per day.

When it is administered topically, compound (I), or a salt, solvate or hydrate thereof, is used at a concentration generally of from 0.001% to 10% by weight, preferably from 0.01% to 5% by weight, relative to the total weight of the composition.

For the treatment of an inflammatory cutaneous pathology, and more particularly eczema or psoriasis, compound (I), or a pharmaceutically acceptable salt thereof or a pharmaceutically acceptable solvate or a hydrate thereof, can also be administered in combination with another active ingredient.

The pharmaceutical and dermatological compositions as described above can therefore contain inert additives, or even pharmacodynamically active additives, or combinations of these additives, and in particular:

wetting agents;

flavor enhancers;

preservatives such as para-hydroxybenzoic acid esters;

stabilizers;

moisture regulators;

pH regulators;

osmotic pressure modifiers;

emulsifiers;

UV-A and UV-B screens;

antioxidants, such as α-tocopherol, butylhydroxy-anisole or butylhydroxytoluene, superoxide dismutase, ubiquinol or certain metal-chelating agents;

depigmenting agents such as hydroquinone, azelaic acid, caffeic acid or kojic acid;

emollients;

moisturizing agents such as glycerol, PEG 400, thiamorpholinone and its derivatives or urea;

anti-seborrhoeic or anti-acneic agents, such as S-carboxymethylcysteine, S-benzylcysteamine, their salts or their derivatives, or benzoyl peroxide;

antibiotics such as erythromycin and its esters, neomycin, clindamycin and its esters, or tetracyclines;

antifungal agents such as ketoconazole or 4,5-poly-methylene-3-isothiazolidones;

agents for promoting hair regrowth, such as minoxidil (2,4-diamino-6-piperidinopyrimidine-3-oxide) and its derivatives, diazoxide (7-chloro-3-methyl 1,2,4-benzo-thiadiazine-1,1-dioxide) and phenyloin (5,4-diphenyl-imidazolidine-2,4-dione);

non-steroidal anti-inflammatories;

carotenoids, and in particular β-carotene;

anti-psoriatic agents such as anthraline and its derivatives;

eicosa-5,8,11,14-tetraynoic acid and eicosa-5,8,11-triynoic acid, their esters and amides;

retinoids, i.e., ligands of RAR or RXR receptors, which may be natural or synthetic;

VDR receptor ligands;

corticosteroids or oestrogens;

α-hydroxy acids and α-keto acids or their derivatives, such as lactic acid, malic acid, citric acid, glycolic acid, mandelic acid, tartaric acid, glyceric acid or ascorbic acid, and also their salts, amides or esters, or β-hydroxy acids or their derivatives, such as salicylic acid and also its salts, amides or esters;

ion channel blockers, such as potassium channel blockers;

or else, more particularly for the pharmaceutical compositions, in combination with medicaments known to interfere with the immune system (for example, cyclosporin, FK 506, glucocorticoids, monoclonal antibodies, soluble receptors, cytokines or growth factors, etc.).

Of course, one skilled in this art will take care to select the possible compound(s) to be added to these compounds in such manner that the desired effect on psoriasis or eczema is not or is not substantially impaired by the envisaged addition.

The study of the properties of the compound of formula (I) has shown that the compound of formula (I), and also the pharmaceutically acceptable salts, solvates or hydrates thereof, are not toxic and have an anti-inflammatory activity in the treatment of eczema and psoriasis, which manifests itself both via topical administration and oral administration.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Compositions

A—Oral Administration

0.2 g tablet:

| | |
|---|---|
| Compound of formula (I) | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

B—Topical Administration:

Ointment:

| | | |
|---|---|---|
| Compound of formula (I) | 0.30 | g |
| White petroleum jelly codex | qs 100 | g |

(b) Lotion:

| | | |
|---|---|---|
| Compound of formula (I) | 0.10 | g |
| Polyethylene glycol (PEG 400) | 69.90 | g |
| 95% ethanol | 30.00 | g |

### EXAMPLE 2

### Evaluation of the Anti-Inflammatory Activity of W3646 (Compound (I)) after Single Topical Application in the Arachidonic Acid-Induced Mouse Ear Edema Test on Balb/c Mice

The arachidonic acid is dissolved in a mixture of THF/methanol at 4%.

Treatment:

The test compound is dissolved in the solution of arachidonic acid (AA) at the final concentration, and tested at concentrations of 0.1, 0.3 and 1%.

20 μl of the solution are applied to the inner face of the right ear.

The thickness of the ear is measured at T+1 h, T+2 h and T+4 h.

Results:

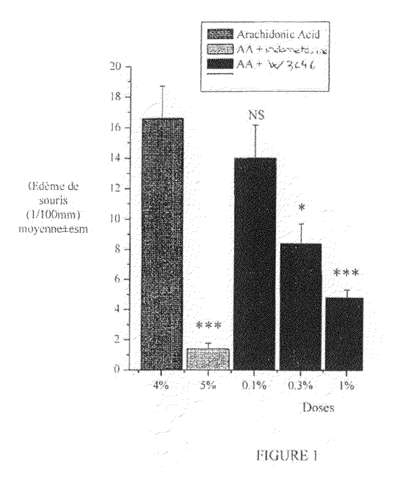

The results are shown in FIG. 1.

One hour after application, the 4% arachidonic acid solution induces an increase in the ear thickness of 76%.

Indomethacin (negative control) at 5% inhibits the ear edema caused by arachidonic acid by 92% (***).

W3646 at 0.1%, 0.3% to 1% reduces the ear edema respectively by 16% (NS), 49% (*) and 71% (**) with a dose-dependent activity.

W3646 therefore shows an anti-inflammatory effect in the arachidonic acid-induced mouse ear edema model.

### EXAMPLE 3

### Evaluation of the Anti-Inflammatory Activity of W3646 (Compound (I)) after Single Topical Application in the TPA-Induced Mouse Ear Edema Test on Balb/c Mice (TPA=Tetradecanoylphorbol Acetate)

Treatment:

The edema is induced by single application of 20 μl of TPA dissolved in acetone at 0.01%.

The test compound is diluted in the TPA solution and applied at a concentration of 0.1%, 0.3% to 1%.

A positive control, β-methasone valerate (VdB), is also tested.

The thickness of the mouse ear is measured at T+6 h.

Results:

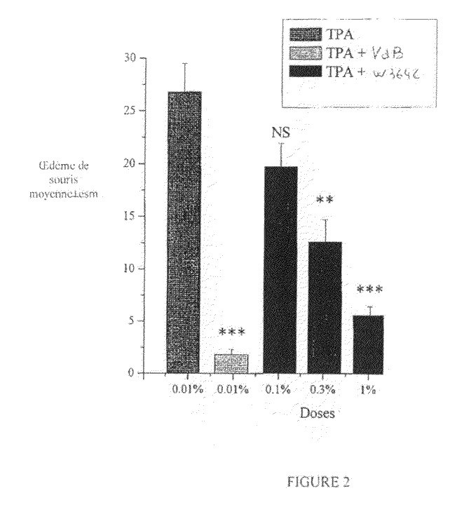

The results are shown in FIG. 2.

After single topical application of the betamethasone valerate positive control (0.01%) diluted in the TPA solution, a reduction in the ear edema of 93% (***) is observed.

W3646 has a strong dose-dependent anti-inflammatory effect, and reduces the TPA-induced ear edema by 26% (NS) (at 0.1%), 53% (**) (at 0.3%) and 79% (***) (at 1%).

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for preventing and/or treating an inflammatory cutaneous pathology/affliction, comprising administering to a subject in need of such treatment, a thus effective amount of at least one compound selected from the group consisting of E-3-[3-[N-(4-methoxybenzenesulfonyl)-N-isopropylamino]-phenyl]-3-(3-pyridyl)propenohydroxamic acid of formula (I): 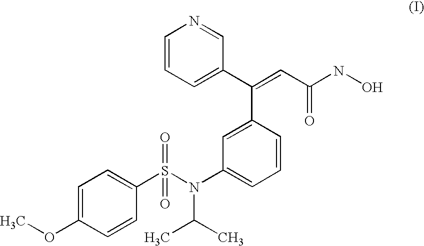
or a pharmaceutically acceptable salt, solvate or hydrate thereof, formulated into a physiologically/pharmaceutically acceptable carrier/excipient therefor.

2. The regime or regimen as defined by claim 1, comprising the treatment of eczema or psoriasis.

3. The regime or regimen as defined by claim 1, said at least one compound being formulated for oral administration.

4. The regime or regimen as defined by claim 1, said at least one compound being formulated for topical administration.

5. The regime or regimen as defined by claim 1, comprising the treatment of cutaneous, mucosal or ungual psoriasis, or psoriatic arthritis.

6. The regime or regimen as defined by claim 1, comprising the treatment of cutaneous or respiratory atopy, or gingival hypertrophy.
